(19) United States
(12) Patent Application Publication
(10) Pub. No.: US 2012/0291669 A1
(43) Pub. Date: Nov. 22, 2012

(54) Sulfosuccinates

(75) Inventors: Stefan Busch, Dusseldorf (DE); Paul Birnbrich, Solingen (DE); Eike Ulf Mahnke, Velbert (DE); Anja Wick, Hilden (DE)

(73) Assignee: Congnis IP Management GmbH, Dusseldorf (DE)

(21) Appl. No.: 13/522,822

(22) PCT Filed: Jan. 14, 2011
(86) PCT No.: PCT/EP2011/000149

§ 371 Date: Jul. 18, 2012

(30) Foreign Application Priority Data
Jan. 23, 2010 (EP) 10000672.5

(51) Int. Cl. C07C 309/70; C08K 5/41; C09D 5/00; C07C 315/02

(52) U.S. Cl. 106/287.24; 558/52; 106/503

(57) ABSTRACT

The invention relates to sulfosuccinates of the general formula (1), where M is hydrogen or a cation and the groups R⁴ and R⁵—independently of each other—are hydrogen or a cation or an alkyl group, wherein no more than one of the groups R⁴ or R⁵ is hydrogen or a cation, wherein said sulfosuccinates contain at least one alcohol building block that is assigned to the mono-alcohols with 8 to 36 carbon atoms in total, on the condition that said mono-alcohols are Guerbet alcohols (GA) containing at least two branches per molecule. Said sulfosuccinates are suitable as surfactants having improved dynamic surface tension.

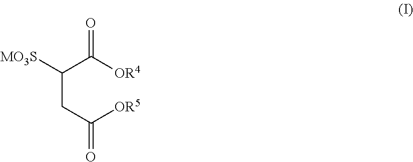

### FIELD OF THE INVENTION

The invention relates to sulfosuccinates of specific structure and compositions comprising these sulfosuccinates and water.

### PRIOR ART

Guerbet alcohols are special branched alcohols. They are primary alcohols which are branched in the 2 position. Guerbet alcohols are known to the person skilled in the art; some have been commercially available for a long time. They are obtained by the so-called Guerbet reaction, a dimerization reaction that has been known for about 100 years which can be outlined by the following equation (R* here means an aliphatic group):

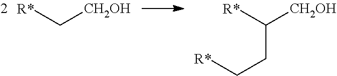

In the course of the classic Guerbet reaction, a primary or secondary alcohol is converted to an alcohol with approximately twice the molecular weight which is alkylated in the 2 position. Thus, for example, n-butanol is converted to 2-ethylhexan-1-ol, hexan-1-ol is converted to 2-butyloctan-1-ol and octan-1-ol is converted to 2-hexyldecan-1-ol.

The primary or secondary alcohols used for the Guerbet reaction carry, on the carbon atom which is directly adjacent to the carbon atom with the OH group, at least one hydrogen atom—in many cases they carry two hydrogen atoms, i.e. a methylene group is then directly adjacent to the carbon atom with the OH group.

The Guerbet reaction typically proceeds in the presence of a base at elevated temperature with the elimination of water and constitutes a simple option for converting linear alcohols to branched alcohols. Typically, only a single alcohol is used in the Guerbet reaction. However, it is also possible to use two different alcohols; in this case, one talks of a mixed Guerbet reaction.

In the equation above, the radicals R* are in each case an aliphatic group. Typically, this is a linear and saturated aliphatic group, i.e. for example fatty alcohols such as octanol or decanol are used as starting alcohols for the Guerbet reaction. From time to time, R* is a saturated cycloaliphatic group, i.e. for example alcohols such as cyclopentanol or cyclohexanol are used as starting alcohols for the Guerbet reaction.

Sulfosuccinates are a surfactant class that has been known for a long time.

As regards the so-called static surface tension, the properties of the sulfosuccinates are determined to a great extent by their alcohol building blocks. As the alkyl radicals of these alcohol building blocks become larger, the ratio of hydrophilic to lipophilic molecular moieties decreases (comparable with the HLB value (hydrophilic-lipophilic balance) proposed by Griffin for nonionic surfactants). Upon varying the size of the alkyl radicals of the alcohol building blocks of sulfosuccinates, an optimum for the surface-active activity is passed through. For example, the maximum achievable reduction in the surface tension of an aqueous solution of didecylsulfosuccinates can be achieved at considerably lower concentrations than with dioctylsulfosuccinates, whereas ditridecylsulfosuccinates again have a considerably weaker surface activity.

A further important parameter for assessing surfactants is the dynamic surface tension. This indicates how rapidly a surfactant can occupy a newly forming surface. This is particularly important in the case of rapid processes such as the application of a coating in the case of printed products. Generally, an increase in the size of the alkyl radical and therefore the increase in size of the surfactant has an unfavorable effect on the dynamic surface tension. On the example of the sulfosuccinates which contain isodecanol and/or 2-propylheptanol as alcohol building blocks, it can be seen that in the case of alcohol building blocks with an identical total number of carbon atoms, the exact structure of the alcohol building blocks has an influence on the dynamic surface tension. In this connection, however, it is the case that a reliable calculation and/or prediction of the dynamic surface tension of a certain surfactant is currently not possible since various processes such as diffusion, adsorption and orientation of the surfactant or certain molecular moieties at the surface have to be taken into consideration very precisely.

Sulfosuccinates can contain linear or branched alcohol building blocks. Commercially used branched alcohol building blocks include methyl-branched isodecanol and isotridecanol and also 2-ethylhexanol. 2-Ethylhexanol, although it is produced industrially by aldol condensation, is formally a Guerbet alcohol because it is accessible by joining two butanol molecules by means of a Guerbet reaction. 2-Propylheptanol is also a Guerbet alcohol because it is accessible by joining two pentanol molecules by means of a Guerbet reaction. However, the sulfosuccinate of 2-propylheptanol has hitherto not been commercially available.

### DESCRIPTION OF THE INVENTION

It was an object of the invention to provide sulfosuccinates which have an improved dynamic surface tension of the corresponding aqueous solutions compared to the sulfosuccinates known from the prior art.

The term “sulfosuccinates” is known to the person skilled in the art. Sulfosuccinates are salts of the following general formula (I)

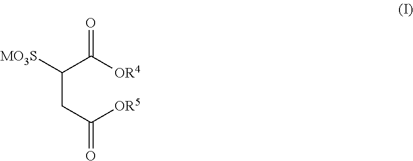

In the formula (I), M is hydrogen or a cation; the radicals R⁴ and R⁵—independently of one another—are hydrogen or a cation or an alkyl group, where at most one of the radicals R⁴ or R⁵ can be hydrogen or a cation. As is known to the person skilled in the art, the term monoalkylsulfosuccinates is used when only one of the radicals R⁴ or R⁵ is an alkyl group, and the term dialkylsulfosuccinates is used when both radicals R⁴ and R⁵ are an alkyl group.

Within the context of the present invention, the term “sulfosuccinates” is used in the aforementioned sense, i.e. it includes both monoalkylsulfosuccinates and also dialkylsulfosuccinates. Since the sulfosuccinates (I) are esters, it is logical to say, if the radicals R⁴ and/or R⁵ are alkyl groups, that the monoalcohols R⁴OH or R⁵OH are the alcohol building blocks of the sulfosuccinates. Use is made below of this simplification of the mode of expression in terms of language.

The present invention firstly provides sulfosuccinates of the general formula (I),

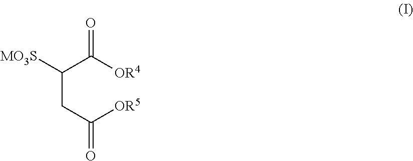

in which M is hydrogen or a cation and the radicals R⁴ and R⁵—independently of one another—are hydrogen or a cation or an alkyl group, where at most one of the radicals R⁴ or R⁵ can be hydrogen or a cation, where these sulfosuccinates contain at least one alcohol building block that is to be assigned to the monoalcohols (i) having in total 8 to 36 carbon atoms, with the proviso that these monoalcohols are Guerbet alcohols (GA) which contain at least two branches per molecule.

In one embodiment, the monoalcohols are those Guerbet alcohols (GA) which contain at least three branches per molecule.

Preferably, the monoalcohols are those Guerbet alcohols (GA) which contain three branches per molecule and which are obtainable by using, as starting alcohols for the

Guerbet reaction for producing these Guerbet alcohols (GA), exclusively those monoalcohols having 4 to 18 carbon atoms which are selected from the group of primary and/or secondary monoalcohols of the formula (MA),

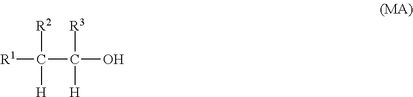

where: (a) the total number of carbon atoms of the compounds (MA) is in the range from 4 to 18; (b) the radicals R¹, R² and R³ are hydrogen or alkyl groups which—independently of one another—may be linear or branched or alicyclic, saturated or unsaturated; (c) the radicals R¹ and R² and/or R¹ and R³ and/or R² and R³ can be joined together, i.e. be part of an alicyclic substructure; (d) the compounds (MA) have at least one branch.

### The Monoalcohols (i)

As stated above, the alcohol building blocks of the sulfosuccinates are monoalcohols (i) having in total 8 to 36 carbon atoms, with the proviso that these monoalcohols are Guerbet alcohols (GA) which contain at least two branches per molecule.

The term branch here is to be understood as follows:
carbon atoms which are joined directly to 3 other carbon atoms (tertiary carbon atoms) are one branch.
carbon atoms which are joined directly to 4 other carbon atoms (quaternary carbon atoms) are two branches.

If the specified condition states that the Guerbet alcohols (GA) must contain at least two branches per molecule, then this means the total number of branches in the molecule.

What Guerbet alcohols and the Guerbet reaction used for their production are is known to the person skilled in the art and important points in this respect have already been explained in the prior art section (see above). As is evident from the equation shown in this connection (see above), the Guerbet reaction consists in the end in joining together two molecules of starting alcohol in a condensation reaction, with the resulting molecule, i.e. the Guerbet alcohol, containing a branch in the molecule at the same position at which the two original starting alcohol building blocks are joined together. If R* in the above equation is a linear aliphatic group, the resulting Guerbet alcohol contains one branch in the molecule. If R* in the above equation is a cycloaliphatic group, the resulting Guerbet alcohol contains two branches in the molecule.

In one preferred embodiment, within the context of the present invention, those Guerbet alcohols (GA) are used which contain at least three branches per molecule.

In a particularly preferred embodiment, within the context of the present invention, those Guerbet alcohols (GA) are used which contain three branches per molecule and which are obtainable by using, as starting alcohols for the Guerbet reaction for producing these Guerbet alcohols (GA), exclusively those monoalcohols having 4 to 18 carbon atoms which are selected from the group of primary and/or secondary monoalcohols of the formula (MA),

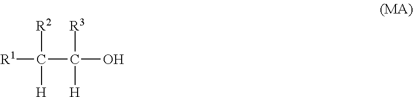

where: (a) the total number of carbon atoms of the compounds (MA) is in the range from 4 to 18; (b) the radicals R¹, R² and R³ are hydrogen or alkyl groups which—independently of one another—may be linear or branched or alicyclic, saturated or unsaturated; (c) the radicals R¹ and R² and/or R¹ and R³ and/or R² and R³ can be joined together, i.e. be part of an alicyclic substructure; (d) the compounds (MA) have at least one branch.

Preferably, here, the alkyl groups of the compounds (MA) are exclusively saturated.

For the compounds (MA) whose alkyl radicals are exclusively saturated, in one preferred embodiment, the total number of their carbon atoms is in the range from 5 to 10. In this connection, alcohols having 5 carbon atoms and in particular isomer mixtures of alcohols having 5 carbon atoms are particularly preferred.

3-Methylbutan-1-ol is very particularly preferred as monoalcohol (MA). It can be used as starting alcohol in pure form or in the form of technical-grade mixtures or else in a mixture with other alcohols of the formula (MA). When using 3-methylbutan-1-ol as monoalcohol (MA), the Guerbet reaction produces the following Guerbet alcohol (GA*)

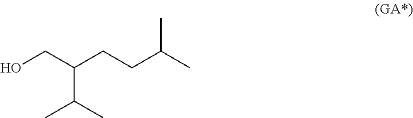

which can be referred to as 2-isopropyl-5-methylhexan-1-ol.

The compounds (MA) can be used in pure form or in the form of technical-grade products. It is also possible to use mixtures for the Guerbet reaction to be carried out according to the invention which essentially contain one or more compounds (MA) as well as further substances—where unbranched aliphatic alcohols are excluded as further substances.

### Further Embodiments

As stated above, M in the formula (I) of the sulfosuccinates has the meaning hydrogen or a cation. Here, the secondary condition that the sulfosuccinates are electrically neutral overall of course applies.

In a preferred embodiment, M is selected from the group of 1-valent cations, in particular from the group of alkali metal cations. Sodium, potassium and ammonium are very particularly preferred as cations M.

As stated above, the sulfosuccinates (I) and the monoalkyl- and/or dialkylsulfosuccinates. Dialkylsulfosuccinates are preferred here.

Dialkylsulfosuccinates in which the monoalcohols (MA) are saturated, at least mono-branched monoalcohols having 5 to 10 carbon atoms, in particular 3-methylbutan-1-ol, are very particularly preferred.

### Compositions

A further subject matter of the invention is compositions comprising one or more of the sulfosuccinates (I) explained in more detail above and water.

### Use

Furthermore, the invention relates to the use of the sulfosuccinates (I) as surfactants. In this connection, the investigations by the applicant have shown that the sulfosuccinates (I) according to the invention are characterized by an improved dynamic surface tension compared to the sulfosuccinates known from the prior art: as is evident by reference to the examples listed below, with the sulfosuccinates according to the invention it is possible to achieve higher surface formation rates without a significant increase in the surface tension.

In further embodiments, the invention relates to the use of the sulfosuccinates (I) as wetting agents, in particular in coatings, and also as emulsifier, in particular in emulsion polymerization.

### EXAMPLES

### Substances Used

| | |
|---|---|
| Sodium disulfite | from BASF |
| Maleic anhydride | from Sasol-Huntsman |
| Hydropalat 875 | anionic wetting agent (from Cognis) |
| C10-V1-OH | 2-propylheptanol (BASF; singly branched alcohol with |
| | 10 carbon atoms) |
| C10-V3-OH | triply branched alcohol with 10 carbon atoms which |
| | has been prepared as follows by Guerbet reaction |
| | from 3-methyl-1-butanol in the presence of catalytic |
| | amounts of aldehyde, alkali and palladium on carbon: |
| | a reaction mixture of 2500 g of 3-methyl-1-butanol |
| | (an isoamyl alcohol), 75 g of 3-methyl-1-butanal, 4 g |
| | of Pd/C (5% Pd/C 3610 from Johnson Matthey in |
| | 50% water) was heated to 180° C., 320 g of KOH |
| | (50% strength) were metered in in portions and run |
| | with a pressure ramp from 4.6 to 1.4 bar for 18 hours |
| | with stirring. The triply branched alcohol obtained is |
| | also referred to below as “isoamyl Guerbet alcohol”. |

### Examples and Comparative Examples

### Example 1 (for Comparison)=B1

### Preparation of di(2-propylheptyl) maleate

In a 2 l four-neck flask with mechanical stirrer, heating, distillation apparatus and nitrogen/vacuum connection, under nitrogen atmosphere, 392 g
(4.0 mol) of maleic anhydride,
1330 g (8.4 mol) of C10-V1-OH (2-propylheptanol) and
8.6 g (0.05 mol) of p-toluenesulfonic acid monohydrate
   were weighed in and slowly heated to 140° C. The water of reaction formed was separated off by distillation. As soon as only a small amount of distillate was still passing over (about 3.5 hours after the start of the reaction), the pressure was slowly reduced to 10 mbar and held for 3 hours. The resulting product had a residual acid number of 1.66 mg KOH/g.

### Example 2 (for Comparison)=B2

### Preparation of sodium di(2-propylheptyl)sulfosuccinate

(Solution in Water/Ethanol)

When carrying out the synthesis according to example 2 for the first time, the procedure was as follows:

In a 11 four-neck flask with mechanical stirrer, heating, reflux condenser and nitrogen line,
574 g (1.45 mol) of di(2-propylheptyl) maleate (prepared according to example 1),
40 g of Hydropalat 875
142 g (0.75 mol) of sodium disulfite and
194 g of demineralized water
   were introduced and the batch was stirred at 104° C. under reflux and a gentle stream of nitrogen until a clear solution had formed (ca. 3 hours). It was then after-stirred for a further 10 minutes. Sulfite could no longer be detected in the product. Upon cooling, the product gelled/solidified, which could be avoided by adding a cosolvent: for this purpose, 50 g of ethanol were added at ca. 80° C.

When carrying out the synthesis according to example 2 for the second time, the procedure was as described above except that instead of 40 g of Hydropalat 875, 40 g of a solution of (a) 70% di(2-propylheptyl) sulfosuccinate, (b) 20% water and (c) 10% ethanol were used (component (a) of this solution was still not available when carrying out the synthesis for the first time as described above).

The product obtained in this way had the following characteristic data: acid number: 0.18 mg KOH/g; Epton: 14.01%; dry residue: 71.66% by weight; content of sodium sulfate: 0.32% by weight.

### Example 3 (According to the Invention)=B3

### Preparation of di(isoamyl Guerbet) maleate

In a 0.5 l four-neck flask with mechanical stirrer, heating, distillation apparatus with water separator and nitrogen/vacuum connection, under a nitrogen atmosphere,
49.03 g (0.5 mol) of maleic anhydride,
162.5 g (1.0 mol) of C10-V3-OH (isoamyl Guerbet alcohol) and
1.04 g (0.01 mol) of p-toluenesulfonic acid monohydrate
   were weighed in and slowly heated to 140° C. The water of reaction formed was separated off by distillation. As soon as only a small amount of distillate was still passing over (about 3.5 hours after the start of the reaction), the pressure was slowly reduced to 10 mbar and held for 3 hours. The resulting product had a residual acid number of 4.26 mg KOH/g.

### Example 4 (According to the Invention)=B4

### Preparation of sodium di(isoamyl Guerbet) sulfosuccinate (aqueous solution)

In a 0.5 l four-neck flask with mechanical stirrer, heating, reflux condenser and nitrogen line,
100 g (0.25 mol) of di(isoamyl Guerbet) maleate (prepared according to example 3),
24.66 g (0.13 mol) of sodium disulfite and
41.31 g of demineralized water
   were introduced and the batch was stirred at 104° C. under reflux and a gentle stream of nitrogen until a clear solution had formed (ca. 3 hours). It was after-stirred for a further 10 minutes. Sulfite could no longer be detected in the product. Upon cooling, the product gelled/solidified.

The product obtained in this way had the following characteristic data: acid number: 3.6 mg KOH/g; Epton: 10.8%; dry residue: 74.5% by weight; content of sodium sulfate: 1.8% by weight.

Dynamic Surface Tension

The dynamic surface tension (ST) was determined by means of bubble pressure tensiometry using a Krüss BP 2 bubble pressure tensiometer. For this purpose, aqueous solutions with an active substance content of 0.1% by weight were prepared; measurement was carried out at 25° C.

The values obtained are listed in table 1. As well as the data for the substance of the comparative example B2 (di-2-PH-SUS) and the data of example B4 according to the invention (di-isoamyl Guerbet-SU), for comparison purposes, additionally the data of two further substances, namely sodium di(2-ethylhexyl)sulfosuccinate (di-2-EH-SUS), which is a tried and tested commercial product, and sodium di(isodecyl)sulfosuccinate (di-isodecyl-SUS), the alcohol building block of which has the same number of carbon atoms (namely ten) as the alcohol building block of the compound according to the invention as per example 4, are given.

TABLE 1

| | | | | | | Di-isoamyl | |
|---|---|---|---|---|---|---|---|
| | | Di-2-PH-SUS | | | | Guerbet-SUS | |
| Di-2-EH-SUS | | (as per example 2) | | Di-isodecyl-SUS | | (as per example 4) | |
| Bubble | Surface | Bubble | Surface | Bubble | Surface | Bubble | Surface |
| frequency | tension | frequency | tension | frequency | tension | frequency | tension |
| [Hz] | [mN/m] | [Hz] | [mN/m] | [Hz] | [mN/m] | [Hz] | [mN/m] |
| 20.112 | 46.1 | 18.311 | 59 | 19.752 | | 19.652 | 53.6 |
| 10.203 | 40.6 | 10.118 | 50.9 | 10.081 | 65.5 | 10.261 | 46.4 |
| 3.062 | 38.8 | 3.065 | 35.9 | 2.967 | 52.9 | 3.062 | 32.4 |
| 2.026 | 38.3 | 2.013 | 32.3 | 2.024 | 49.1 | 2.016 | 30.2 |
| 0.507 | 36.1 | 0.523 | 27.0 | 0.5 | 31.7 | 0.52 | 28.0 |
| 0.256 | 35.2 | 0.254 | 26.3 | 0.249 | 29.2 | 0.25 | 27.6 |
| 0.098 | 33.8 | 0.099 | 26.1 | 0.093 | 26.9 | 0.086 | 27.1 |

It was found that the sulfosuccinate according to the invention as per example 4 on the one hand effectively reduces the static surface tension (ST) (the ST of the di-isoamyl Guerbet-SUS according to the invention at bubble frequencies below 0.1 Hz is significantly lower than the ST of the commercial product di-2-EH-SUS), and on the other hand has a surprisingly good dynamic behavior. Thus, the ST of aqueous solutions of di-isoamyl Guerbet-SUS (whose alcohol building block has 10 carbon atoms and is triply branched) increases noticeably only at bubble frequencies above 2 Hz and increases greatly only above 3 Hz. In the case of di-2-PH-SUS (whose alcohol building block likewise has 10 carbon atoms, but is only singly branched), this is observed at just 0.5 and, respectively, 2 Hz. Comparison with the commercial product di-2-EH-SUS (whose alcohol building block has 8 carbon atoms and is singly branched) moreover reveals that the increase in the ST with increasing bubble frequency firstly turns out to be less than in the case of the di-isoamyl Guerbet-SUS according to the invention, although it then has a sharp increase in the ST at a bubble frequency of 10 Hz. On account of the considerably lower static ST of the sulfosuccinate according to the invention, the ST of its aqueous solution only exceeds that of an aqueous di-2-EH-SUS solution (di-C8V1-SUS) at a bubble frequency of 5 Hz. Since the bubble formation frequency is a measure of the rate of new surface formation, it can be established that the sulfosuccinates according to the invention permits an improvement in the technical applications described above.

1. A sulfosuccinate of the general formula (I), 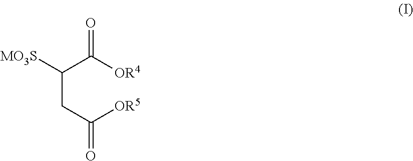
wherein M is hydrogen or a cation and the radicals R⁴ and R⁵ are each independently hydrogen or a cation or an alkyl group, where at most one of the radicals R⁴ or R⁵ is hydrogen or a cation, wherein these sulfosuccinates contain at least one alcohol building block comprising a monoalcohol having in total 8 to 36 carbon atoms, with the proviso that these monoalcohols are Guerbet alcohols (GA) which contain at least two branches per molecule.

2. The sulfosuccinate as claimed in claim 1, wherein the monoalcohols are Guerbet alcohols (GA) which contain at least three branches per molecule.

3. The sulfosuccinate as claimed in claim 2, wherein the monoalcohols are Guerbet alcohols (GA) obtained by exclusively using a monoalcohol having 4 to 18 carbon atoms as starting alcohols in a Guerbet reaction, wherein the monoalcohol is which are selected from the group of primary and/or secondary monoalcohols of formula (MA), 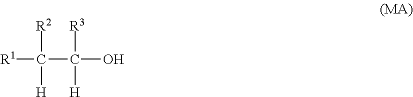
where: (a) the total number of carbon atoms of the compounds (MA) is in the range from 4 to 18; (b) the radicals R¹, R² and R³ are hydrogen or alkyl groups which—independently of one another—may be linear or branched or alicyclic, saturated or unsaturated; (c) the radicals R¹ and R² and/or R¹ and R³ and/or R² and R³ can be joined together, (d) the compounds (MA) have at least one branch.

4. The sulfosuccinate as claimed in claim 3, wherein the monoalcohols (MA) are saturated, at least monobranched, monoalcohols having 5 to 10 carbon atoms.

5. The sulfosuccinate as claimed in claim 3, wherein the monoalcohol (MA) is 3-methylbutan-1-ol.

6. The sulfosuccinate as claimed in claim 1, wherein M is a 1 valent cation.

7. The sulfosuccinate as claimed in claim 6, wherein M is an alkali metal cation.

8. The sulfosuccinate as claimed in claim 6, wherein M is an ammonium ion.

9. The sulfosuccinate as claimed claim 1, wherein the sulfosuccinate is a dialkylsulfosuccinate.

10. A composition comprising one or more sulfosuccinates as claimed in claim 1 and water.

11. A method of making a coating, the method comprising adding a sulfosuccinates (I) as claimed in claim 1 solution as a wetting agent.

12. The method as claimed in claim 1, wherein the radicals R¹ and R² and/or R¹ and R³ and/or R² and R³ are part of an alicyclic substructure.

13. The sulfosuccinate of claim 1, wherein the monoalcohol has a formula represented by R⁴OH or R⁵OH.

14. The sulfosuccinate as claimed in claim 2, wherein M is a 1-valent cation.

15. The sulfosuccinate as claimed in claim 14, wherein M is an alkali metal cation.

16. The sulfosuccinate as claimed in claim 14, wherein M is an ammonium ion.

17. A method of making a sulfosuccinate of claim 1, the method comprising reacting di(isoamyl Guerbet) maleate and sodium disulfate.
